(19) United States
(12) Patent Application Publication
(10) Pub. No.: US 2002/0098228 A1
(43) Pub. Date: Jul. 25, 2002

(54) Transdermal drug delivery device

(75) Inventors: Ezri Tarazi, Shoham (IL); Shai Baruch Goitein, Herzlia Pituach (IL); Eric J. Messika, Rehovot (IL); Michal Ghozi, Rehovot (IL); Max Herzberg, Moshav Satariya (IL)

Correspondence Address: G.E. EHRLICH (1995) LTD., c/o ANTHONY CASTORINA, SUITE 207, 2001 JEFFERSON DAVIS HIGHWAY, ARLINGTON, VA, 22202, US

(73) Assignee: TRANSDERMICS LTD.

(21) Appl. No.: 09/767,854

(22) Filed: Jan. 24, 2001

(51) Int. Cl. A61K 9/70

(52) U.S. Cl. 424/449

(57) ABSTRACT

A transdermal drug delivery device in the form of a patch having an outer wall including a piercing element on its inner face facing the drug reservoir and effective, when depressed towards the opposite side of the patch, to pierce a sealing film and thereby to permit the delivery of the drug transdermally to the user. The outer wall of the patch is of a stiff elastic material which produces an audible “click” when depressed from its outer position to its inner position piercing said sealing film. In one described embodiment, the outer wall is of a monostable construction, and in a second described embodiment, it is of a bistable construction.

### FIELD AND BACKGROUND OF THE INVENTION

The present invention relates to transdermal drug delivery devices, and particularly to a patch for application to the skin of a user for delivering drugs through the user's skin.

The delivery of drugs through the user's skin provides many advantages over the conventional oral or injection techniques of drug administration. Thus, the transdermal delivery of drugs is generally more comfortable and convenient to the user, avoids possible gastrointestinal irritation, and enables the drug administration to be immediately terminated should a need arise, e.g., in the event of an adverse drug reaction. As examples of various types of transdermal drug delivery devices that have been developed, reference is made to U.S. Pat. Nos. 4,911,707; 4,917,676; 4,956,181; 5,008,110; 5,066,494; 5,271,940; 5,505,958; 5,641,507; 5,846,559 and 5,968,536.

### OBJECTS AND BRIEF SUMMARY OF THE PRESENT INVENTION

An object of the present invention is to provide a transdermal drug delivery device of a novel construction having a number of important advantages, as will be described more particularly below.

According to one aspect of the present invention, there is provided a transdermal drug delivery device, comprising a patch for application to the skin of a user; the patch including a drug reservoir for receiving a quantity of a drug to be delivered transdermally to the user, and an outer wall on one side of the patch, which outer wall is finger-depressible from an outer position towards the opposite side of the patch to an inner position, in order to deliver drug to the user from the drug reservoir through the opposite side of the patch: the opposite side of the patch including a sealing film normally sealing the drug within the drug reservoir; the outer wall including a piercing element on its inner face facing the drug reservoir and effective, when the outer wall is depressed towards the opposite side of the patch from the outer position of the wall to its inner position, to pierce the sealing film, and thereby to permit the delivery of the drug transdermally to the user through the opposite side of the patch.

According to a further feature in the described preferred embodiment, the piercing element has a rounded tip to minimize accidental piercing of the sealing film.

According to another aspect of the present invention, there is provided a transdermal drug delivery device comprising a patch for application to the skin of a user; the patch including a drug reservoir for receiving a quantity of a drug to be delivered transdermally to the user, and an outer wall on one side of the patch, which outer wall is finger-depressible from an outer position towards the opposite side of the patch to an inner position, in order to deliver drug to the user from the drug reservoir through the opposite side of the patch: the outer wall being of a stiff elastic material which produces an audible “click” when depressed from its normal outer position to its inner position delivering drug from the drug reservoir to the user through the opposite side of the patch.

In one described preferred embodiment, the outer wall is of a monostable construction and automatically returns to its outer position upon release after its depression. In a second described preferred embodiment, it is of a bistable construction and remains in its inner position upon release after its depression.

As will be described more particularly below, transdermal drug delivery devices can be constructed in accordance with the foregoing features having a simple and efficient structure producible in volume and at low cost, and conveniently useable by the users.

Further features and advantages of the invention will be apparent from the description below.

### BRIEF DESCRIPTION OF THE DRAWINGS

The invention is herein described, by way of example only, with reference to the accompanying drawings, wherein:

FIG. 1 is a sectional view illustrating one form of transdermal drug delivery device constructed in accordance with the present invention;

FIG. 2 is an enlarged perspective view, partly in section, more particularly illustrating the construction of the device of FIG. 1;

FIGS. 3a-3c illustrate three stages in the use of the device of FIGS. 1 and 2;

FIG. 4 is a sectional view illustrating a second form of transdermal drug delivery device constructed in accordance with the invention, the device being shown in its stable normal or initial condition; and

FIG. 5 illustrates the device of FIG. 4 in its stable actuated condition.

### DESCRIPTION OF PREFERRED EMBODIMENTS

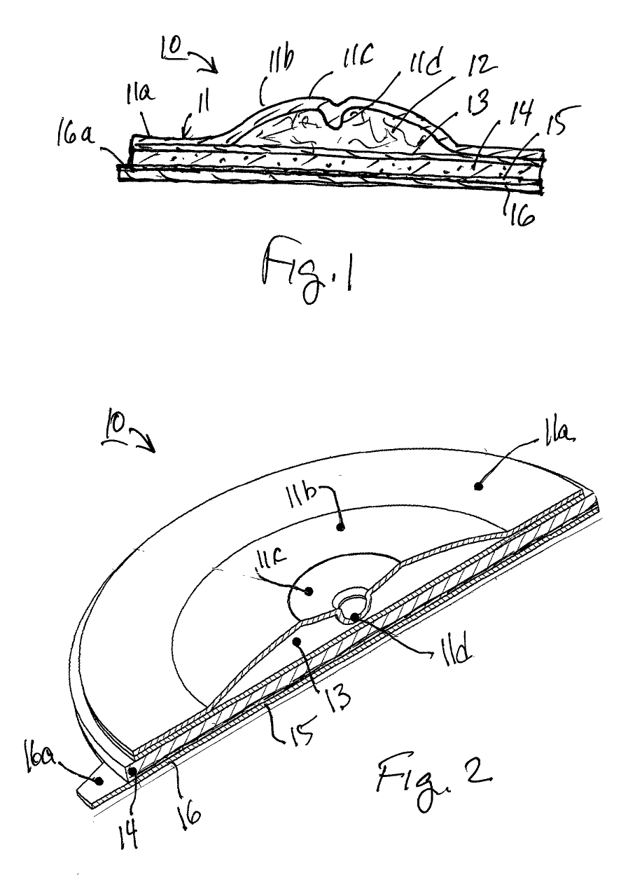

### The Embodiments of FIGS. 1-3c

FIGS. 1 - 3c illustrate one form of transdermal drug delivery device in the form of a skin patch, generally designated 10, to be applied to the skin of the user. The illustrated patch includes the following elements: an outer wall 11 defining one side of a drug reservoir 12 for receiving a quantity of the drug to be delivery transdermally to the user; a sealing film 13 defining the opposite side of the drug reservoir 12; an absorbent pad 14 on the outer side of the sealing ring 13; an adhesive layer 15 on the outer side of the pad 14; and a removable protective liner 16 covering the adhesive layer 15. All the foregoing elements are of circular configuration such that the produced patch 10 is of circular configuration, except that the removable protective liner 16 may be provided with a projecting tab, as shown at 16a in FIG. 2, to facilitate removal of the liner when the patch is to be applied to the skin of a user.

As also shown in FIG. 2, the outer wall 11 is integrally formed with an outer annular margin 11a, and a central dome-shaped region 11b. The center of the dome-shaped region 11b is made transparent, as shown at 11c, to enable the user to view the drug reservoir 12 of the patch. In addition, the central region 11c of the outer wall is formed with an inwardly-directed projection 11d which serves to pierce the sealing film 13 when the dome-shaped section 11b of the outer wall is pressed inwardly, as will be described more particularly below. In order to minimize accidental piercing of the sealing film 13, projection 11d is rounded such that a significant intentionally-applied force is required to cause the projection lid to pierce the sealing film 13.

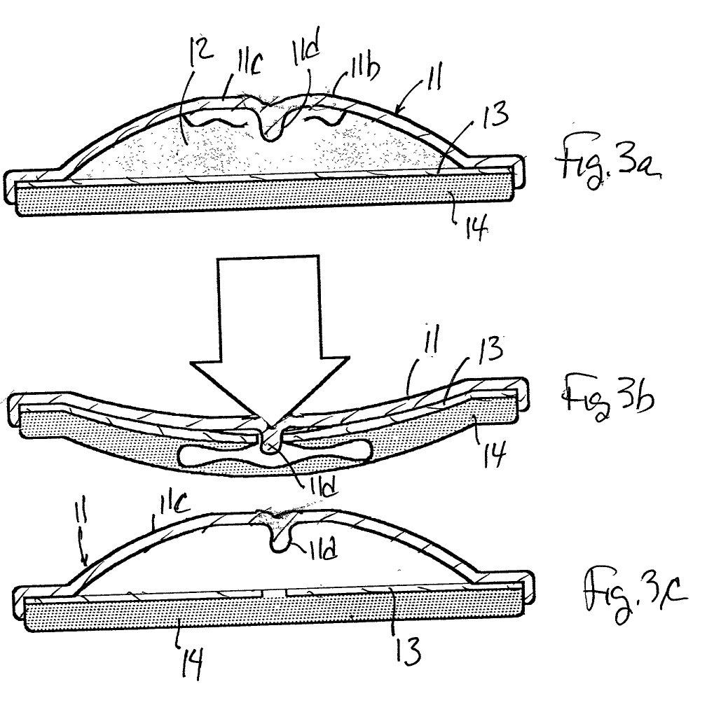

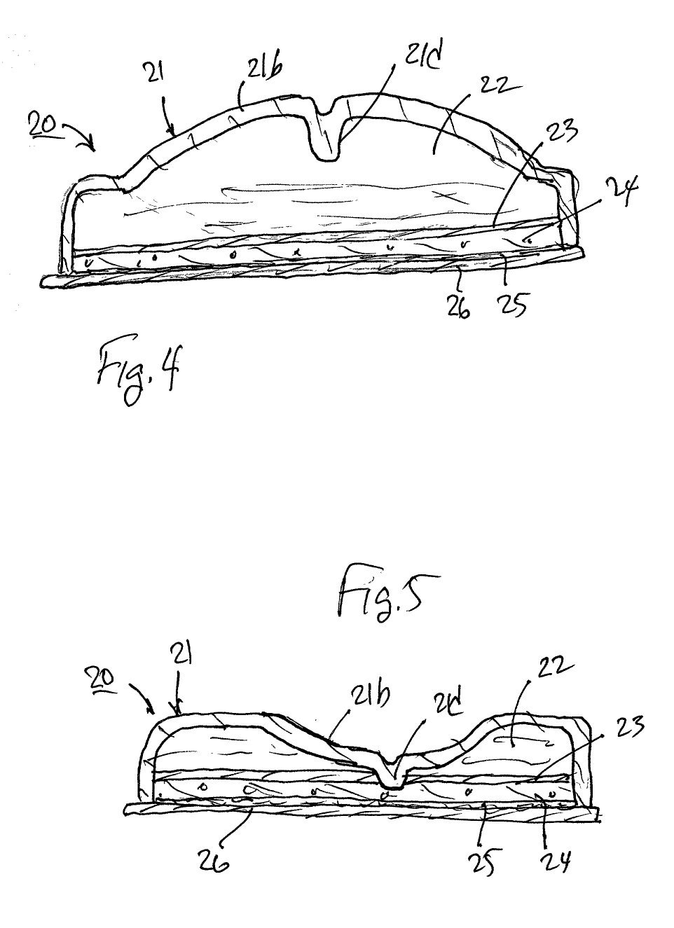

Outer wall 11 is made of a stiff elastic plastic material which produces an audible “click” when the dome-shaped section 11b is depressed from its outer position as shown in FIGS. 1, 2 and 3a, to its inner position, shown in FIG. 3b, where its projection 11c pierces the sealing film 13. Such a “click” thus provides an audible signal to the user that the sealing film 13 has been pierced, and therefore the drug within the reservoir 12 can flow therefrom to the absorbent pad 14.

FIGS. 3a-3c illustrate the manner of using the patch 10 of FIGS. 1 and 2.

Thus, when the patch is to be used, the protective liner 16 is removed, e.g., by pulling tab 16a, thereby exposing the adhesive layer 15. The patch is then applied to the user's skin by pressing adhesive layer 15 against it, which adhesive layer retains the patch in place.

When it is desired to produce a transdermal delivery of the drug within reservoir 12 to the user, the dome-shaped section 11b of the outer wall 11 is pressed inwardly by the user's finger, as shown in FIG. 3b. This causes its piercing element 11d to pierce the sealing film 13. The drug within reservoir 12 is thereby exposed to the absorbent pad 14. Pad 14 absorbs the drug from the reservoir, and delivers the drug to the user's skin to which the pad is adhered by the adhesive layer 15. As the dome-shaped section 11b of the outer wall 11 is pressed to pierce the sealing film 13, it produces a “click” which can be heard by the user to indicate to the user that the sealing film has been pierced, and therefore the patch has been activated to deliver the drug to the person's skin via absorbent pad 14.

As shown in FIG. 3c, when the user releases the dome-shaped section 11b of the outer wall 11, that section will snap-back to its outer position, as shown in FIG. 3c. The construction illustrated in FIGS. 1 and 2 is thus a monostable construction; that is, the wall can assume only one stable state, namely the outer, normal position as illustrated in FIGS. 3a and 3c.

If the user wishes to accelerate the feeding of the drug from the reservoir 12 to the absorbent pad 14, this may be done by repeatedly depressing and releasing the dome-shaped section 11b of the outer wall 11.

FIG. 4 illustrates a transdermal drug delivery patch, therein generally designated 20, of the same construction as described above with respect to FIGS. 1-3c, including an outer wall 21, a drug reservoir 22, a sealing film 23, an absorbent pad 24, an adhesive layer 25 and a protective layer 26, except that the outer wall 21 is of a bistable construction, rather than a monostable construction. Thus, when the outer wall 21 is of a bistable construction, its depressible dome-shaped section 21b is capable of assuming two stable states, namely the outer initial state as illustrated in FIG. 4, or an inner, actuated state as illustrated in FIG. 5. Thus, as shown in FIG. 5, after the dome-shaped section 21b has been depressed from its outer initial position as illustrated in FIG. 4 to its inner actuated position illustrated in FIG. 5 wherein its projection 21d has pierced the sealing film 23, the wall will remain in the latter position upon release of the outer wall section 21b.

Such a construction may be desirable to accelerate the flow of the drug from the reservoir 22 to the absorbent pad. This would occur because of the continuous application of the pressure of the outer wall section 21b pressing the drug in reservoir 22 towards the absorbent pad 24 in the actuated condition of the outer wall. In addition, this bistable operation of the patch 20 illustrated in FIG. 4 provides a quick indication of whether or not the patch has been actuated.

It will be appreciated that the patch illustrated in FIG. 4 is otherwise constructed, and operates, in the same manner as described above with respect to FIGS. 1-3c, in that patch 20 of FIGS. 4 and 5 also provides a “click” when the wall section 21b passes from one bistable condition to the other.

While the invention has been described with respect to two preferred embodiments, it will be appreciated that these are set forth merely for purposes of example, and that many other variations, modifications and applications of the invention may be made.

What is claimed is:

1. A transdermal drug delivery device, comprising a patch for application to the skin of a user;
said patch including a drug reservoir for receiving a quantity of a drug to be delivered transdermally to the user, and an outer wall on one side of the patch, which outer wall is finger-depressible from an outer position towards the opposite side of the patch to an inner position, in order to deliver drug to the user from said drug reservoir through said opposite side of the patch:
said opposite side of the patch including a sealing film normally sealing the drug within the drug reservoir;
said outer wall including a piercing element on its inner face facing the drug reservoir and effective, when said outer wall is depressed towards said opposite side of the patch from the outer position of the wall to its inner position, to pierce said sealing film, and thereby to permit the delivery of the drug transdermally to the user through said opposite side of the patch.

2. The device according to claim 1, wherein said piercing element has a rounded tip to minimize accidental piercing of the sealing film.

3. The device according to claim 1, wherein said outer wall is of a stiff elastic material which produces an audible “click” when depressed from its outer position to its inner position piercing said sealing film.

4. The device according to claim 3, wherein said outer wall is of a monostable construction and automatically returns to its outer position upon release after its depression.

5. The device according to claim 3, wherein said outer wall is of a bistable construction and remains in its inner position upon release after its depression.

6. The device according to claim 3, wherein said outer wall includes a central dome-shaped section which is depressible to produce the audible “click”.

7. The device according to claim 3, wherein said outer wall includes a transparent section which enables viewing the drug-reservoir.

8. The device according to claim 1, wherein said opposite side of the patch further includes an absorbent pad having an outer surface to be placed in contact with the user's skin, said sealing film being carried on the inner surface of the absorbent pad facing said drug reservoir.

9. The device according to claim 8, wherein the patch further includes a removable protective liner over said outer surface of the absorbent pad.

10. The device according to claim 8, wherein the patch further includes an adhesive coating over said outer surface of the absorbent pad.

11. A transdermal drug delivery device, comprising a patch for application to the skin of a user;
said patch including a drug reservoir for receiving a quantity of a drug to be delivered transdermally to the user, and an outer wall on one side of the patch, which outer wall is finger-depressible from an outer position towards the opposite side of the patch to an inner position, in order to deliver drug to the user from said drug reservoir through said opposite side of the patch:
said outer wall being of a stiff elastic material which produces an audible “click” when depressed from its normal outer position to its inner position delivering drug from the drug reservoir to the user through said opposite side of the patch.

12. The device according to claim 11, wherein said out wall is of a monostable construction and automatically returns to its outer position upon release after its depression.

13. The device according to claim 11, wherein said outer wall is of a bistable construction and remains in its inner position upon release after its depression.

14. The device according to claim 11, wherein said outer wall includes a central dome-shaped section which is depressible to produce the audible “click”.

15. The device according to claim 11, wherein said outer wall includes a transparent section which enables viewing the drug-reservoir.

16. The device according to claim 11, wherein:
said opposite side of the patch includes a sealing film normally sealing the drug within the drug reservoir; and
said outer wall includes a piercing element on its inner face facing the drug reservoir and effective, when said outer wall is depressed towards said opposite side of the patch from the outer position of the wall to its inner position, to pierce said sealing film, and thereby to permit the delivery of the drug transdermally to the user through said opposite side of the patch.

17. The device according to claim 16, wherein said piercing element has a rounded tip to minimize accidental piercing of the sealing film.

18. The device according to claim 11, wherein said opposite side of the patch further includes an absorbent pad having an outer surface to be placed in contact with the user's skin, said sealing film being carried on the inner surface of the absorbent pad facing said drug reservoir.

19. The device according to claim 18, wherein the patch further includes a removable protective liner over said outer surface of the absorbent pad.

20. The device according to claim 18, wherein the patch further includes an adhesive coating over said outer surface of the absorbent pad.
